(19) United States
(12) Patent Application Publication
(10) Pub. No.: US 2007/0043268 A1
(43) Pub. Date: Feb. 22, 2007

(54) Guided Electrical Transcranial Stimulation (GETS) Technique

(76) Inventor: Michael J. Russell, Davis, CA (US)

Correspondence Address: Attn: Jeffry S. Mann, Ph.D., J.D.;Morgan Lewis & Bockius, LLP, Spear Street Tower, One Market, San Francisco, CA, 94105, US

(21) Appl. No.: 11/424,813

(22) Filed: Jun. 16, 2006

Related U.S. Application Data
(60) Provisional application No. 60/691,068, filed on Jun. 16, 2005.

(51) Int. Cl. A61B 5/00

(52) U.S. Cl. 600/300

(57) ABSTRACT

An optimal transcranial or intracranial application of electrical energy for is determined for therapeutic treatment. MRI or CAT scan data, or both, are obtained for a subject brain. Different electrical resistance values are assigned to portions of the subject brain based on the data. Electrode sites are selected. Based on the assigning and selecting, one or more applied electrical inputs are calculated for optimal therapeutic application of transcranial or intracranial electricity.

### CROSS-REFERENCE TO RELATED APPLICATIONS

This application claims the benefit of priority to U.S. provisional patent application 60/691,068, filed Jun. 16, 2005, which is hereby incorporated by reference.

### BACKGROUND

1. Field of the Invention

The invention relates to guided electrical transcranial stimulation, or GETS, and particularly to accurately assigning resistivities to current-carrying organic material in and around the brain, and to determine optimal application of electrical inputs such as current, voltage, charge, or power, including any of various pulse characteristics such as pulse duration and number of pulses per pulse trains, for medical treatment.

2. Description of the Related Art

The advent of transcranially stimulated electrical motor evoked potentials (tcMEPs) has resulted in a dramatic reduction in the rate of paralysis for high risk surgical patients (see Chappa K H, 1994, Calanchie et al 2001, Pelosi et al. 2002, Bose B, Sestokas A K, Swartz D M 2004 and MacDonald et al 2003, citations below and hereby incorporated by reference). As a consequence tcMEPs have become the standard of care for testing the integrity of the cortical spinal track during spinal and neurosurgical procedures. Unfortunately, transcranial electrical stimulation has generally required high voltages with diffuse current spread that causes the activation of large regions of the brain and puts the patient at risk of unwanted and unknown side effects. Obtaining more precisely directed current at lower voltages will reduce the risk and greatly expand the utility of transcranial stimulation for surgical and non-surgical patents.

It is desired to have a technique involving site specific transcranial electrical stimulation of the brain that approximates physiological current densities, and to apply these techniques to treat expanded patient populations, including spinal surgery patients. Transcranial electrical stimulation to elicit motor evoked potentials (tcMEPs) has become the standard of care for monitoring the motor pathways of the spinal cord and brain during high risk surgeries. A conventional tcMEP technique can often be a crude, but effective tool to monitor motor pathways and to identify iatrogenic injuries. FIG. 1A illustrates a tcMEP from a scoliosis patient. The scale of FIG. 1A shows 50 μV on the y axis and 7.5 ms on the x-axis. Applied pulses were 150 Volts for 100 μs in trains of five pulses with ISI of 3 ms. FIG. 1B illustrates a tcMEP from a 86 year old male with a neck fracture. Applied pulses were 75 Volts in the upper plot and 25 Volts in the lower plot.

Typically, a tcMEPs procedure involves placing electrodes in the patient's scalp at locations that are thought to encompass the motor cortex and then applying brief high voltage electrical pulses with the intention of activating distal muscles or muscle groups. FIG. 2 illustrates placement of electrodes J₀ outside of a patient's scalp. FIG. 2 also illustrates three regions S₀, S₁, and S₂ having different conductivities σ₁, σ₂, and σ₃, respectively. Unfortunately, the high voltages typically used to induce tcMEPs and the responses they produce can activate whole regions of the head, body, or trunk as well as the target muscles. The movement of large muscle groups due to the uncontrolled current spread means that seizures, broken jaws and patient movement create risk factors that have been associated with tcMEP testing (see Chappa, K H, 1994, citation below). Applying stimulus trains rather than single pulses and adjustments in anesthesia techniques have significantly reduced the applied electrical currents used from 700-900 V to 200-400 V (see Chappa, K H. 1994, Haghighi S S, and Zhange R 2004, citations below and hereby incorporated by reference).

TcMEPs have become widely accepted as a less onerous substitute for “wake-up tests” in which the patient is awakened during surgery and asked to move their limbs before the surgical procedure is completed (see Eroglu, A et al. 2003, citation below and hereby incorporated by reference). However, these reduced stimulus levels still exceed normal physiological levels and the uncontrolled movement of large muscle groups suggests that the applied pulses continue to result in significant current spreads. While major side effects are relatively rare, tongue lacerations, muscle tears, and bucking are still rather common side effects (see Calanchie, B et al. 2001, citation below and hereby incorporated by reference). The large muscle movements that are sometimes associated with tcMEPs also limit the usefulness of the tcMEPs during periods when the surgeon is involved in delicate brain or spinal procedures.

It is desired to reduce or eliminate these side effects by predicting the paths of electrical pulses within the brain and consequently adjusting current levels (i.e., lower). It is also desired to reducing the current strength to near physiological levels at targeted areas to allow brain electrical stimulation to be used for treatment of patients outside of surgery. In this way, a significant positive impact on the treatment of a number of disease conditions that have been demonstrated to benefit from brain electrical stimulation, e.g., Parkinson's disease, chronic pain, and depression, can be achieved.

### Modeling

The head is a heterogeneous, anisotropic conductive medium with multiple conductive compartments. Finding the current path through this medium has been a significant problem in neurophysiology. For decades it has been the dream of many investigators to stimulate the brain through this medium without the use of brain surgery or depth electrodes. It is desired to model and test an innovative solution to this problem.

There is a volume of literature attempting to model current pathways and tissue resistivity that was developed for understanding the source generators of electroencephalography (EEG) (see Rush S, Driscoll D A 1968, Vauzelle, C., Stagnara 1973, Henderson, C J, Butler, S R, and Class A, 1978, citations below and hereby incorporated by reference). This is the inverse problem in that the investigators were trying to determine the source of electrical currents from the brain based on surface recording. In the inverse problem, estimations of source location are made from calculations of a best fit between the measured EEG and potentials modeled using the source parameters and head electrical properties. They have often been used to localize generators or model skull defects for scalp recorded EEG (Benar & Gotman, 2002; Henderson et al., 1975; and Kavanaugh et al., 1978, citations below and hereby incorporated by reference). In the GETS (guided electrical transcranial stimulation) model, the forward problem is addressed for determining optimal current paths from known or selected sources placed on the scalp, and assuming no internal sources. The forward problem is inherently easier in that the conductivity distribution and current source locations are known.

Several authors have attempted to construct such physical models of the head. Some of these physical models were made of plastic, saline and/or silicon. They are not sufficient to represent the complexity of the problem and do not allow for individual differences in anatomy.

Finite element (FE) forward modeling has benefited from recent improvements in estimates of skull and tissue resistivity. These newer estimates were obtained in vivo (see Goncalves et al., 2003; and Oostendorp et al., 2000, citations below and hereby incorporated by reference). These provide more precise values of indigenous tissues than many of the previous estimates that were typically done on dried or cadaver tissues.

Several groups have attempted to resolve the problem of transcranial stimulation by using commercially available transcranial magnetic stimulators. Although magnetic stimulators are commonly used in clinics, they have been rejected for surgical applications because of the difficulty in using them in an environment with multiple metal objects and their tendency for the stimulation parameters to be less consistent than those produced by electrical stimulation. Small movements of the magnetic pulse generators have resulted in significant changes in the stimulus parameters and the coil cannot be used for chronic conditions wherein treatment would involve continuous stimulation. It is desired to accurately model head tissues and current pathways to more efficiently target cerebral activation of corticospinal tract neurons by transcranial electrical stimulation.

### SUMMARY OF THE INVENTION

A technique is provided for determining an optimal transcranial or intracranial application of electrical energy for therapeutic treatment. MRI or CAT scan data, or both, are obtained for a subject brain and/or another body tissue. Different anisotropic electrical values are assigned to portions of the subject brain or other body tissue based on the data. Electrode sites are selected. Based on the assigning and selecting, one or more applied electrical voltages, powers, energies, currents or charges are calculated for optimal therapeutic application of transcranial or intracranial current, or trans-tissue current for other body tissues. The brain is generally referred to herein as a specific tissue with which the invention and embodiments may be advantageously applied, but it is understood that the invention may be applied to other body tissues besides the brain.

The assigning may include segmenting the subject brain by defining tissue compartment boundaries between, and one or more electrical characteristics to, said portions of the subject brain, implementing a finite element model by defining a mesh of grid elements for the subject brain, and ascribing vector resistance values to each of the grid elements based on the segmenting. The segmenting may include discriminating two or more of cerebral spinal fluid, white matter, blood, skin, gray matter, soft tissue, cancellous bone, eye fluid, cancerous tissue, inflammatory tissue, ischemic tissue, and compact bone. The discriminating may involve resolving peaks within respective gray scale data corresponding to the two or more organic brain substances. The ascribing may involve inferring anisotropies for the resistance values of the grid elements.

The “electrical values” may include conductivities, resistivities, capacitances, impedances, or applied energies, or combinations thereof. “Electrical characteristics” may include characteristics relating to conductivities, resistivities, capacitances, impedances, or applied energies, or combinations thereof. “Resistance values” may include resistivities or conductivities or both. The data may include a combination of two or more types of MRI or CAT scan data, or both, such as two or more of T1, T2 and PD MRI data. The data is preferably three-dimensional data.

The selecting may include in preferred embodiments disposing the electrodes on the surface of the skin, in or below the skin (subdermal), or within the skull tissue, and in alternative embodiments, disposing the electrodes through the skull proximate to or in contact with the dura, or at a shallow transdural location. In the alternative embodiments, the selecting may include utilizing a screw mounted electrode within or through the skull tissue.

A further technique is provided for determining an optimal transcranial or intracranial application of electrical energy for therapeutic treatment. A combination of two or more types of three-dimensional MRI or CAT scan data, or both, is obtained for a subject brain. Different electrical values are assigned to portions of the subject brain based on the data. In this embodiment, electrode sites are selected including disposing at least one electrode at least partially through the skull. Based on the assigning and selecting, one or more applied electrical inputs, such as voltage, energy, power, charge, or electrical pulses or pulses trains of selected duration, height, or number, or combinations thereof, are calculated for optimal therapeutic application of transcranial or intracranial electricity, preferably in the form of current.

The assigning may include segmenting the subject brain by defining tissue compartment boundaries between, and one or more anisotropic electrical resistance characteristics to, said portions of the subject brain, implementing a finite element model by defining a mesh of grid elements for the subject brain, and ascribing vector resistance values to each of the grid elements based on the segmenting. The segmenting may include discriminating two or more of cerebral spinal fluid, white matter, blood, skin, gray matter, soft tissue, cancellous bone, eye fluid, cancerous tissue, inflammatory tissue, ischemic tissue, and compact bone.

The data may include a combination of two of more of T1, T2 and PD MRI data. The selecting may include disposing at least one electrode through the skull proximate to or in contact with the dura, or in a shallow transdural location. The selecting may involve utilizing a screw mounted electrode within or through the skull tissue.

A further technique is provided for determining an optimal transcranial or intracranial application of electrical energy for therapeutic treatment. MRI or CAT scan data, or both, are obtained for a subject brain and/or other body tissue. The subject brain or other body tissue is segmented by defining tissue compartment boundaries between, and one or more electrical characteristics to, said portions of the subject brain of other body tissue. A finite element model is implemented by defining a mesh of grid elements for the subject brain of other body tissue. Electrical values are ascribed to each of the grid elements based on the segmenting. Electrode sites are selected. Based on the assigning and selecting, one or more applied electrical inputs, such as voltage, energy, power, charge, or electrical pulses or pulses trains of selected duration, height, or number, or combinations thereof, are calculated for optimal therapeutic application of transcranial or intracranial electricity, preferably in the form of current.

The electrical values preferably include vector resistance values and the electrical characteristics preferably include anisotropies.

The segmenting may include discriminating two or more of cerebral spinal fluid, white matter, blood, skin, gray matter, soft tissue, cancellous bone, eye fluid, cancerous tissue, inflammatory tissue, ischemic tissue, and compact bone. The ascribing may include inferring anisotropies for the resistance values of the grid elements. The data may include a combination of two or more types of MRI or CAT scan data, or both, such as a combination of two of more of T1, T2 and PD MRI data. The data may include three-dimensional data.

A method is further provided for determining an optimal transcranial or intracranial application of electrical energy for therapeutic treatment based on MRI or CAT scan data, or both, of a subject brain and/or other body tissue, and different anisotropic electrical values assigned to portions of the subject brain based on the data. The method involves selecting electrode sites, and calculating, based on the assigned anisotropic electrical values and the selecting, one or more applied electrical inputs, such as voltage, energy, power, charge, or electrical pulses or pulses trains of selected duration, height, or number, or combinations thereof for optimal therapeutic application of transcranial or intracranial electricity, preferably in the form of current.

The anisotropic values are preferably assigned based on segmenting the subject brain by defining tissue compartment boundaries between, and one or more electrical characteristics to, said portions of the subject brain and/or other body tissue, implementing a finite element model by defining a mesh of grid elements for the subject brain, and ascribing vector electrical values to each of the grid elements based on the segmenting. The segmenting may involve discriminating two or more of cerebral spinal fluid, white matter, blood, skin, gray matter, soft tissue, cancellous bone, eye fluid, cancerous tissue, inflammatory tissue, ischemic tissue, and compact bone. The discriminating may involve resolving peaks within respective gray scale data corresponding to two or more brain or other body tissues.

A further method is provided for determining an optimal transcranial or intracranial application of electrical energy for therapeutic treatment based on obtaining MRI or CAT scan data, or both, of a subject brain and/or other body tissue, and electrical values ascribed to grid elements of a mesh defined by implementing a finite element model for a subject brain, and by segmenting the subject brain and/or other body tissue by defining tissue compartment boundaries between, and one or more electrical characteristics to, said portions of the subject brain and/or other body tissue, and by implementing a finite element model by defining a mesh of grid elements for the subject brain and/or other body tissue, and ascribing electrical values to each of the grid elements based on the segmenting. The method includes selecting electrode sites, and calculating, based on the ascribed electrical values and selecting, one or more applied electrical inputs, such as voltage, energy, power, charge, or electrical pulses or pulses trains of selected duration, height, or number, or combinations thereof for optimal therapeutic application of transcranial or intracranial electricity, preferably in the form of current.

The electrical values may be as defined above, and may preferably include vector resistance values, while the electrical characteristics may be as defined above, and preferably include anisotropies. The segmenting may include discriminating two or more of cerebral spinal fluid, white matter, blood, skin, gray matter, soft tissue, cancellous bone, eye fluid, and compact bone. The ascribing may include inferring anisotropies for the resistance values of the grid elements.

One or more processor readable storage devices are also provided having processor readable code embodied thereon. The processor readable code is for programming one or more processors to perform any of the methods recited or described herein for determining an optimal transcranial or intracranial application of electrical energy for therapeutic treatment.

### BRIEF DESCRIPTION OF THE DRAWINGS

FIG. 1A illustrates a tcMEP from a scoliosis patient.

FIG. 1B illustrates a tcMEP from a 86 year old male with a neck fracture.

FIG. 2 illustrates a human head with materials of different conductivities conventionally identified and having two electrodes coupled therewith.

FIG. 3 illustrates a human brain having a mesh for finite element modeling applied thereto.

FIG. 4 illustrates a human brain having several tissue compartments identified and segmented in accordance with a preferred embodiment.

FIG. 5 illustrates a human brain having several tissue compartments having different anisotropic resistivities identified and segmented, and having a mesh for anisotropic finite element modeling applied thereto.

FIG. 6a illustrates a human brain with two selected electrode locations and a current path defined therein.

FIG. 6b illustrates the human brain of FIG. 6a having a mesh for finite element modeling applied thereto.

FIG. 6c illustrates the human brain of FIG. 6b with anisotropies ascribed to elements of the mesh.

FIG. 6d shows plots of current density through identical regions of isotropic and anisotropic models.

FIG. 7a illustrates current density variations around areas of varying anisotropic resistivities.

FIG. 7b illustrates a finite element mesh with mesh elements of different sizes and shapes.

FIG. 8 illustrates MRIs of three different types: T1, T2 and PD.

FIG. 9 illustrates a MRI and a plot of resistivities of tissues showing multiple resolved peaks achieved by gray scale differentiation of tissues of different resistivities.

FIG. 10 illustrates three-dimensional modeling of current densities applied to a human brain coupled with two electrodes.

FIGS. 11a-11d illustrate electrode configurations in accordance with alternative embodiments.

### DETAILED DESCRIPTION OF THE PREFERRED EMBODIMENTS

### Abreviations

CT=Computer Tomography x-ray
GETs=Guided Electrical Transcranial stimulation
EEG=Electroencephalogram
MRI=Magnetic Resonance Imaging
FE=Finite Element method of matrix algebra
SEP=Somatosensory Evoked Potentials
fMRI=functional Magnetic Resonance Imaging
tcMEP=transcranial Motor Evoked Potentials

### Introduction

As will be described in more detail below, solutions to the forward problem are achievable with matrix algebra by constructing a model of sufficient detail representing all the heterogeneities found within an individual's head and brain. The approach described below in the Detailed Description section has bypassed the use of a physical model and uses an individual's MRI and/or CT scan as a representation of the head and brain. MRIs and CT scans are digitized images that can be manipulated through computer programs to which standard algebraic manipulations can be applied. This digital modeling also allows the use of matrix algebra solutions that have been developed for other complex representations e.g. weather systems, fluid streams, etc. Further, modules within finite element (FE) analysis packages have been developed to represent time dependent factors such as capacitance and resistance.

It is further described below to advantageously reduce current densities by utilizing 3-D modeling of the head. Our pilot work has demonstrated that the 2-D Guided Electrical Transcranial stimulation (GETs) developed in our laboratory is able to reduce current densities by 60 percent or more. Greater reduction is achieved with the 3-D model.

Effective embodiments are provided including combining CT scans with MRI images. Such combinations can be advantageously utilized as a base for a GETs model. Computer Tomography (CT) is a particularly effective method of modeling bone and is utilized in embodiments further enhancing the GETs model.

In one embodiment, direct measurements are obtained of current within subject brains. In another embodiment, motor evoked potentials are obtained as a biological assay. A technique in accordance with a preferred embodiment works advantageously in reducing electrical current densities even when brain anatomy has been significantly altered by an injury, tumor, or developmental disorder.

In addition, GETs MODELING can be applied to actual spinal surgery patients. This can serve to optimize transcranial stimulation of the motor cortex.

### Preliminary Studies

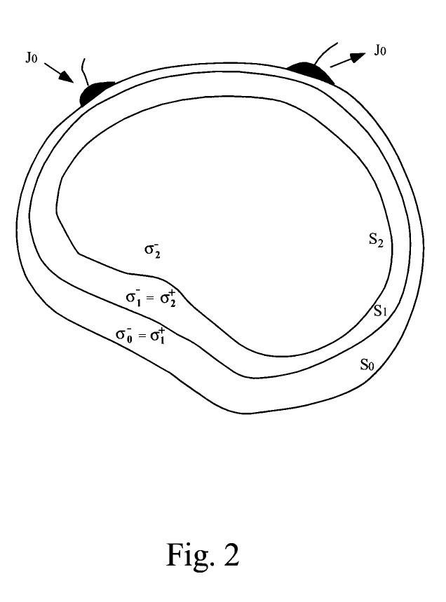

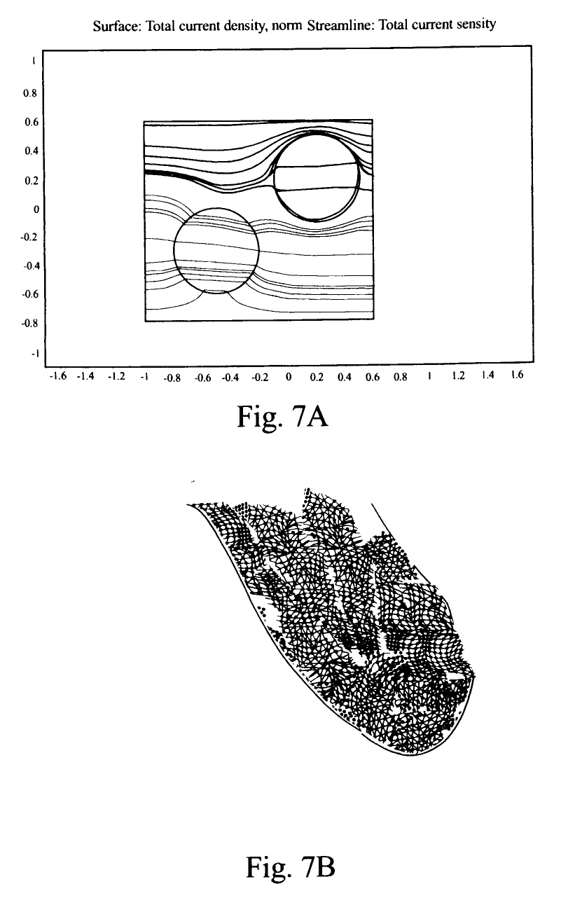

In pilot work to the preferred embodiment which involves three-dimensional modeling, a two dimensional (2-D) model has been developed of a single MRI slice through a head, in accordance with an alternative embodiment. FIG. 3 illustrates a human brain having a mesh for finite element modeling applied thereto (see also FIG. 7B which illustrates a finite element mesh with mesh elements of different sizes and shapes). The mesh includes elements of different shapes and sizes that have different resistivities assigned to them. In the 2-D embodiment, current paths after transcranial stimulation can be predicted, e.g., in an anatomically correct coronal section through the upper limb representation of motor cortex, using FEM methods.

Current densities are obtained in this embodiment for a coronal MRI section (6.5 mm) through the upper limb motor cortex. The modeling procedes in two steps: segmentation to identify tissue compartment boundaries and resistivities, and then implementation of a finite element model to solve the forward problem (modeling measurements using given parameter values) for current densities.

### Segmentation

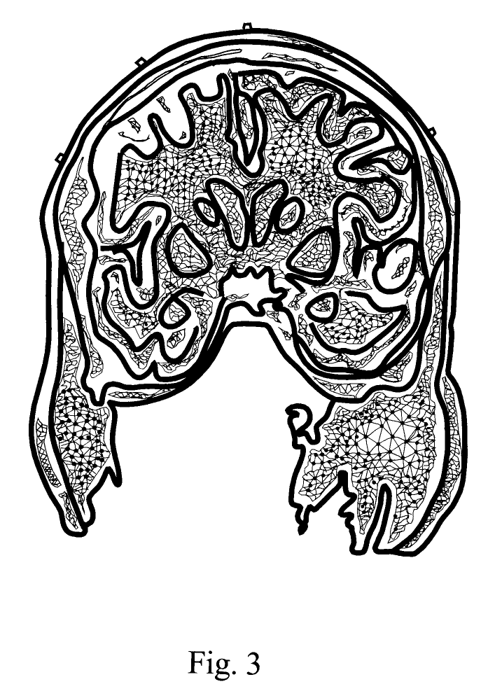

The scanned image is preferably contrast enhanced and then preliminary tissue compartment boundaries are identified automatically, semi-automatically or manually, and preferably using commercially available software (e.g., Canvas). FIG. 4 illustrates a human brain having several tissue compartments identified and segmented according to their different resistivities in accordance with a preferred embodiment. The tissue compartments that are segmented in the representation of FIG. 4 include cerebral spinal fluid (CSF) at 65 ohm-cm, white matter at 85 ohm-cm, blood at 160 ohm-cm, skin at 230 ohm-cm, gray matter at 300 ohm-cm, soft tissue at 500 ohm-cm, cancellous bone at 2500 ohm-cm, and compact bone at 16000 ohm-cm.

Most of the tissue resistivity estimates were taken from Haueisen et al. (1997), which summarized resistivity values from many studies and provided mean values for tissue compartments. The exception is the resistivity for white matter, which was taken from the summary of Geddes and Baker (1967). We used a longitudinal (as compared to transverse) estimate obtained from the internal capsule of the cat (Nicholson, 1965). A longitudinal estimate is appropriate because this is the dominant orientation of fibers for a small electrode positioned tangential to a site on cerebral cortex. As mentioned before the values for bone were taken from Goncalves et al., 2003; Oostendorp et al., 2000.

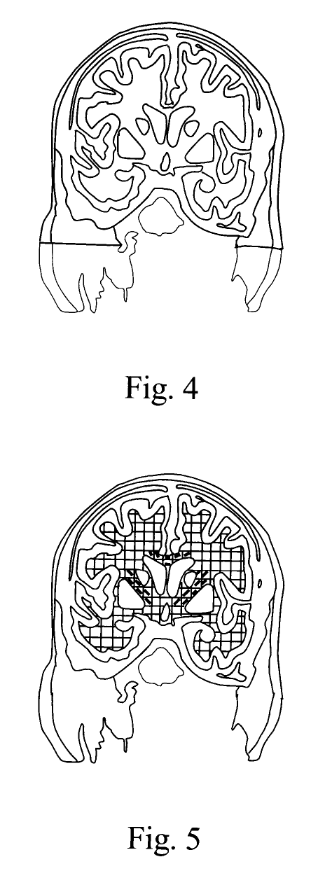

The preliminary boundaries are then superimposed over an original MRI, such as the MRI illustrated in FIG. 5. Final segmentation of tissue compartments may be completed by hand. Matching MRI and anatomical sections from human brain atlases of Talairach and Tournoux, and Schaltenbran and Wahren (Nowinski et al., 1997, citation below and hereby incorporated by reference) greatly aided in identifying gray matter compartments, particularly deep brain nuclei.

In FIG. 5, a grid is shown which serves as a finite element mesh, and the elements have directionalities or anisotropies ascribed thereto and illustrated with the slanted lines inside the elements of the grid. These directionalities correspond to directionalities of the nerve fibers.

### Identifying Tissue Resistivities Based on MRI Data

A relationship of tissue resistivity to MRI gray scale that can be correlated to tissue types can be expressed by the formula:
R(V)=K(1−v)^{E}+D, where

R=Resistivity;

V=Numeric value of MRI data*;

K=Multiplier value;

E=Exponent; and

D=Density value.

*The V value can be either simple MRI data values or combined values from multiple MRIs or multiple types of MRIs. Exemplary values include K=1600, E=4 and D=65.

Anisotropies/directionalities can be inferred from the anatomy or determined based on the MRI data, or a combination thereof. A direct determination is accomplished by diffusion tensor MRI (DT-MRI, or DTI). The indirect is accomplished by inferring the direction of fibers, specifically nerve fibers, by the general anatomy. DT-MRI data are sometimes called Anisotrophic MRIs.

### Finite Element Modeling

The pilot alternative embodiment 2-D current densities are expressed as amps per meter, while the preferred embodiment three-dimensional 3-D current densities are expressed in amps per square centimeter that would be applied in a 3-D model. Units of coulombs per square centimeter may also be used for modeling pulses.

Bilateral electrode placements (and an applied potential difference of 100 V) are calculated for the segmented section, using a FE model generated using FEMLAB (Comsol Pty Ltd, Burlington Mass.). A mesh may be constructed by first detecting edge contours of each segment within the image, then converting the region within each contour into 2 D subdomains. Meshing of the entire structure may be carried out using standard FEMLAB meshing routines, requiring that minimum element quality be 0.1, (quality parameter varies between 0 and 1, acceptable minimum mesh quality is 0.6). The modal value of mesh quality is preferably around 0.98. Triangle quality is given by the formula:
q=4√3a÷[h₁²+h₂²+h₃²], where
a is the triangle area and h₁, h₂, and h₃ are side lengths of the triangle; and q is a number between 0 and 1. If q>0.6, the triangle is of acceptable quality, and q=1 when h₁=h₂=h₃. If triangle elements have low q they are typically long and thin, which may result in the solution on the mesh being inaccurate.

The linear meshes for the model illustrated at FIG. 3 contained approximately 180,000 elements and 364,000 degrees of freedom. Solution of the models to a relative precision of less than 1×10−6 involved around 27 s on a Dell Workstation (2.4 GHz processor, 2 GB RAM) running Linux (RedHat 3.0 WS).

### Results

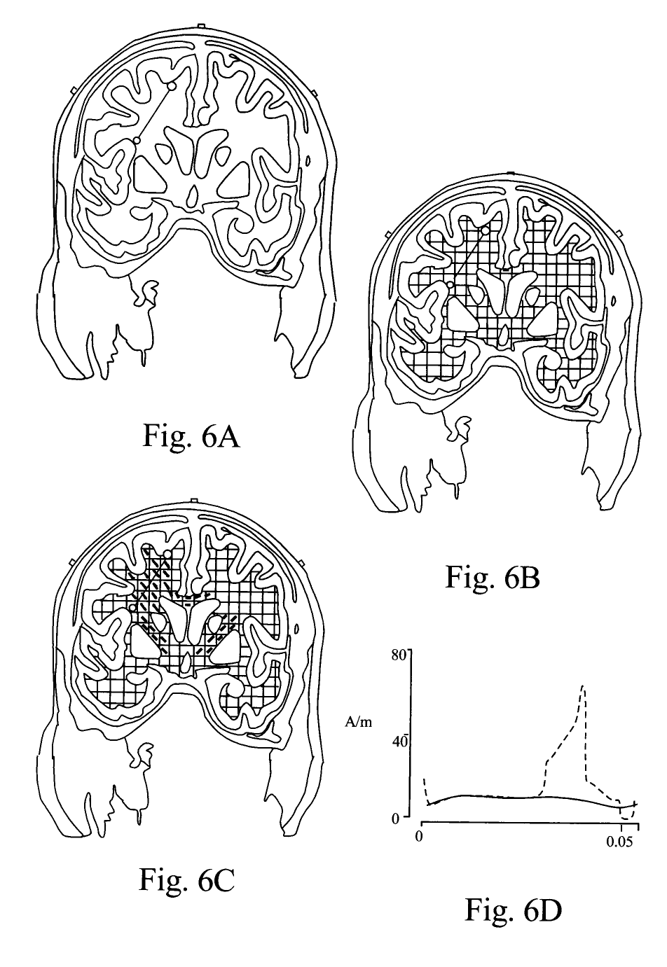

The modeling results are illustrated at FIGS. 6A-6D. The image of FIG. 6A was calculated without adjusting to the anisotropic properties of the white matter. The image FIG. 6A includes a representation of a human brain with multiple compartments segmented by values of resistivity and having line boundaries. There are also illustrated a pair of electrode locations “+” and “−”. A current path of interest CPI is also indicated in FIG. 6A.

The image of FIG. 6B has a matrix or grid of squares, rectangles, or other polygons such as triangles over it. The image of FIG. 6B differs from that of FIG. 6A because it is adjusted for directionality of current flow through nerves or anisotropy. FIG. 6C illustrates the anisotropies taken into account in the FIG. 6B representation by having directional lines within at least some of the polygons that make up the grid. Striking differences are illustrated at locations of current density “hot spots” within the central regions of the brain near the ventricles. Tissue anisotropy has a significant influence on the location of these hot spots.

The line plots in FIG. 6D are of current densities through identical locations along the current path of interest CPI illustrated at FIGS. 6A, 6B and 6C. The solid line IM in FIG. 6D is the current density for the isotropic model represented at FIG. 6A, while the dashed line AM in FIG. 6D is the current density for the more realistic anisotropic model of FIGS. 6B and 6C. A peak P around 68 A/m was observed for the anisotropic model, while the isotropic model provided a maximum of 16 A/m for the homogeneous white matter region studied along the CPI.

The GETs model demonstrates some expected and unexpected results. As expected, there is a concentration of current below the electrodes. However, the optimal current path demonstrated is not always the path of least resistance. There are regions of high current density where there is a high conductivity inclusion within a sphere of lower conductivity (see red zones at the pituitary stalk and the ventricle) (see Knudsen 1999 and Grimnes, S. and Martinsen O. G. 2000, citations below and hereby incorporated by reference, for detailed explanations of why this occurs). FIG. 7A illustrates this effect. The effect appears to create hot spots of electric field induced in the surrounding low conductivity region. The current increase is greatest in the vicinity of interfaces that lie perpendicular to the current flow. Some of these current densities are substantially above the surrounding area and significantly distant to the placement of the electrodes. In this context, the challenge is to determine electrode locations such that unwanted activation is minimized, while stimulating targeted areas efficiently.

Tissue anisotropy is advantageously modeled in accordance with a preferred embodiment, and it has been modeled for an injection current in the brain. Models of further embodiments include anisotropic modeling of blood vessels and directionality of muscle fibers. Because the GETs model is based on MRIs and/or CAT scans of individuals, it also adjusts to developmental and individual differences in brain structure. Among the most significant of these are the differences in bone structure.

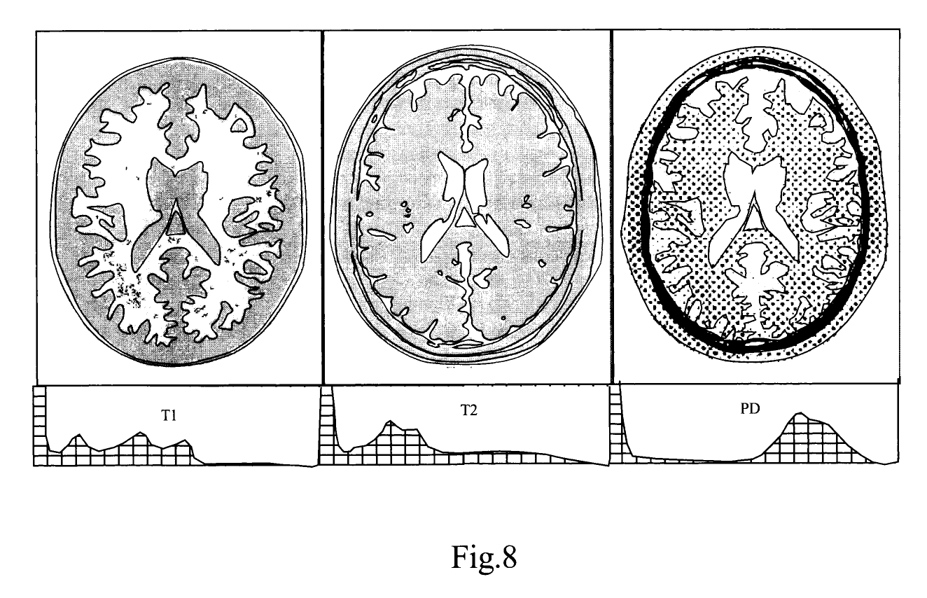

FIG. 8 illustrates MRIs of three different types: T1, T2 and PD. Below each MRI is a gray scale. The gray scale for the T1 MRI appears to resolve three peaks which may correspond to three distinct tissue types having three different resistivities. The gray scale for T2 shows one, or possibly two, peaks, and the gray scale for PD shown one peak at a different resistivity than T2 or T1. By utilizing information from different MRI types, it is possible to enhance gray scale segmentation.

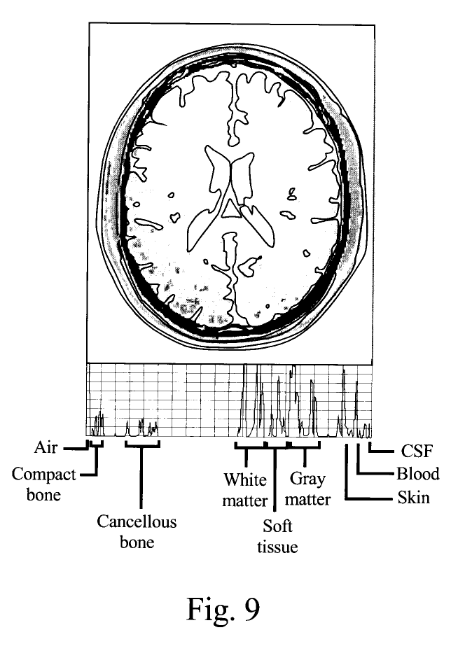

FIG. 9 illustrates a MRI and a plot of resistivities of tissues showing multiple resolved peaks achieved by gray scale differentiation of tissues of different resistivities. Advantageously in accordance with a preferred embodiment, the gray scale for the MRI shown in FIG. 9 resolves multiple peaks corresponding to various tissue types including compact bone, cancellous bone, white matter, soft tissue, gray matter, skin, blood and cerebral spinal fluid. Other resolvable tissues may include cancerous tissue, inflammatory tissue and ischemic tissue, as well as eye fluid. By having enhanced resolution of tissues, it is possible to assign more correctly the vector resistivities or other electrical values to brain or other body tissues, and thereby calculate more precisely the optimum current or other electrical input to be applied for therapeutic treatment, e.g., for chronic pain among other ailments.

### Individual Differences and Developmental Variations

Bone is the highest resistivity tissue in the body thus making the skull a significant barrier to injection currents. There are also considerable variations in skull thickness and density between sites within and between individuals. The cranial sutures, penetrating vessels and individual anomalies provide low resistivity paths through the skull that are important sources of individual variation.

Developmentally, the presence of highly vascularized fontanel in young children provides a path for current through the skull, because of the fontanel's much lower resistivity (scalp: 230 Ωcm; blood: 160 Ωcm; bone 7560 Ωcm) compared with the surrounding bone. These fontanels are substantially closed by 1.5 years to form the sutures present in the adult skull (Law, 1993, citation below and incorporated by reference). The sutures remain open for some time in many adults, and do not close at all in some aged individuals, although in others they close completely. By adjusting for these differences rather than simply increasing the current, we are able to significantly reduce currents needed to stimulate the brain of an individual.

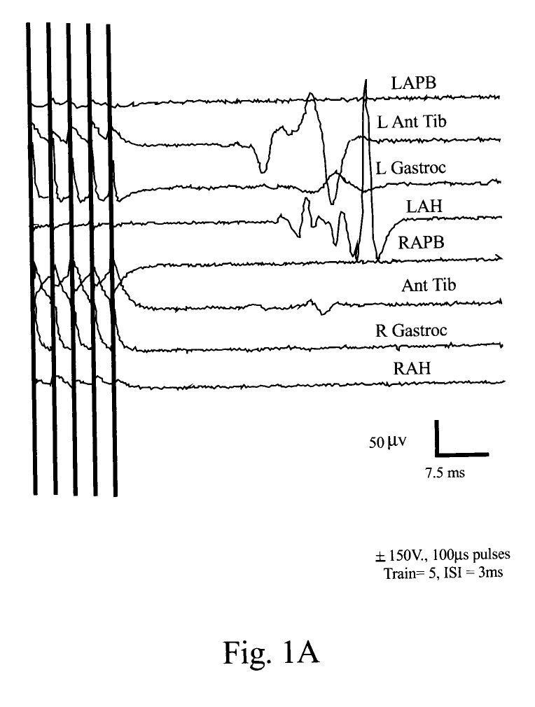

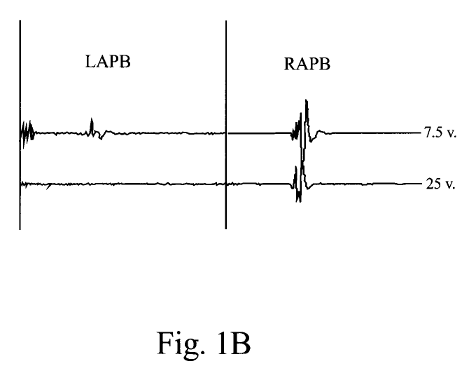

FIGS. 1A and 1B were introduced earlier. FIG. 1A shows MEPs evoked by transcranial stimulation in a 14 year old scoliosis patient. The electrode positions were approximately at C1 and C2 (10-20 system), with anodal stimulation applied at C2 (50V). The largest amplitude MEPs were evoked from muscles of the left foot (abductor hallucis) and leg (anterior tibialis), although smaller responses from the abductor hallucis muscle on the right side was also noted. No responses were recorded in the abductor pollicic brevis muscles of either hand. These relatively low current responses were obtained by slight adjustments in electrode locations. Similar adjustments varying from patient to patient may be used to optimize MEP signals.

In alternative embodiments, it is possible to reduce the level of stimulation for intraoperative monitoring and improve our understanding of what is occurring with tcMEP. In preferred embodiments, however, significant further improvement is achieved. Additional improvements are provided in the model by: 1) utilizing a three-dimensional GETs model; 2) improving the detail in the images to account for blood vessels, finer nerve tracks and bone anomalies; 3) adding into the model the effects of capacitance found at tissue boundaries; 4) verifying the model with direct brain measurements; or 5) by applying findings to the motor cortex in refractory Parkinsonism patients, or combinations thereof.

### Research Design and Methods

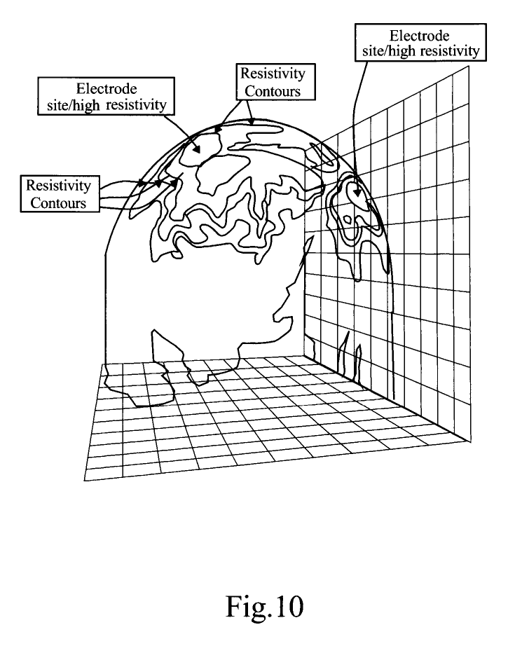

In one embodiment, GETs models are provided in 3-D, and finer detail is applied to the images, while effects of capacitance are added which involves a conversion from resistivity to impedance. FIG. 10 illustrates three-dimensional modeling of current densities applied to a human brain coupled with two electrodes. FIG. 10 shows contours of constant resistivity or voltage drop. FIG. 10 illustrates the high resistivity around the electrodes and changing resistivities along any current path that traverses multiple tissues. Existing 3-D MRI images of two normal adult brains may also be used. In one embodiment, the images are segmented, a FE mesh is generated, and then the analysis is performed for isotropic models and/or anisotropic models with and without capacitance. Capacitance may be an important factor as membrane capacitance at tissue boundaries as well as a significant factor in determining stimulus tissue penetration (see Grimnes S. Martinsen O. G 2000, citation below and incorporated by reference).

### Segmentation

Segmentation, or the outlining, identifying, ascribing and/or assigning of resistivity values to MRI slices in 3-D, can be a difficult and arduous task. The effort involved may be significantly reduced by commercial automated tissues analysis algorithms and services. One or these, Neuroalyse, Inc (Quebec, Canada) may be preferably selected to perform such analysis. This system can perform more than 90% of the tissue segmentation and leave blank the areas of the tissue that the software is unable to resolve or where it is preferred to more particularly work with these areas. This automated segmentation is particularly advantageous as new MRI images have 2 mm thicknesses and record in three planes. The results are checked and any blank areas filled in by hand or other precision automation, or otherwise. Tissue resistivities are assigned preferably as above, except tissue slices are preferably finer and values are preferably included for blood vessels and skull sutures. Resulting 2-D sliced images are then interleaved into a three 3-D model. A final 3-D segmentation and meshing may be performed using AMIRA (Mercury Computer Systems, Berlin, Germany) and the resulting 3-D models generated may be imported into Femlab (Comsol, Burlington Mass.) for FE calculation.

The 3-D images, with identified motor cortex, may be analyzed using the FE method. To identify the best sites for stimulation, an additional analysis may be performed by iteratively moving representative paired electrode locations across the scalp and evaluating effects at the target site (motor cortex). This targeting may be performed by having the computer systematically select and test for the highest current density at the target site for each of the locations of the traditional 10-20 system for electrode placements as current injection and extraction sites with a constant current pulse. In addition to the traditional 10-20 system, sites that may be considered or selected may include eye lids, auditory canals and nasal passages as these additional locations represent avenues for bypassing the high resistivity of the skull bone. After the computer has grossly identified a pair of stimulation and extraction sites, the model may be refined by testing in one centimeter increments around selected sites of the 10-20 system.

These predicted “best fit” locations may then be tested against the two “standard” locations most commonly presented in the current literature (C3-C4 and Cz′-FPz of the 10-20 system) (see Deletis, 2002 and MacDonald et al. 2003, citations below and incorporated by reference). This 3-D effort provides an advantageously sophisticated model, although verification and human testing are preferably still used, as well.

In a further embodiment, the technique includes 1) adding CT scans to MRI images, 2) verifying the GETs model with two assays and testing the models in surgical subjects, and/or 3) applying the model to spinal surgery patients. MRI's are effective at imaging soft tissue, but are less effective at imaging bone, because of the dependence of MRI's on water molecules within the target tissues. The bony skull is the highest resistivity tissue in the head and a significant barrier for electric current passing into the brain. Our modeling has compensated for this by assuming that dark regions between the brain and the scalp are bony structures. This can have the advantage of only obtaining only a single scan of a patient, as long as the quality remains high. Test the efficacy of adding CT scans to GETs may be performed with MRI's and combined MRI/CTs. The MRIs may be 2 mm scans from a 1.5 Tesla magnet collected in three axes (axial, coronal, and sagital). The CT images may be scanned at 2.5 mm and retroactively adjusted to match the three axes of the MRI scans. The two sets of images may then be digitally co-registered and segmented, e.g., as above. This combined imaging may be performed on ten patients who are scheduled for ventricular shunts. The data from these patients may then be GETs modeled both with the simple MRI and the combined MRI/CT scans as data sets. These same patients may then be tested for current density during tcMEP stimulation.

### Direct Measurement

Currents may be directly measured in the cerebral ventricle of patients who are about to have a ventricular drain placed in their brain for elective shunt placement for hydrocephalus. In this clinical procedure, a small craniotomy is performed, the dura is then opened, and one end of a silastic tube is placed through the brain and into the ventricle for the purpose of draining excess cerebral spinal fluid. This sylastic tube is filled with saline or cerebral spinal fluid to avoid bubbles and used as a drain. Thus, a saline filled tube can act as a recording electrode placed in the ventricle and passing through brain tissues. Record from this tube may be performed by inserting a platinum/iridium probe in the distal end of the tube and connecting the probe to a recording oscilloscope. After the oscilloscope is turned on, three sets of transcranial pulses will be applied to the patient and the pulsed current measured from the ventricular space will be measured. To reach the ventricle, the tube is placed through a section of prefrontal cortex and readings are taken in this region as well. The readings for the current levels in the sampled regions may be compared to the current levels predicted by the GETs model. The sylastic ventricular drain tube itself has resistivity and capacitance properties and these may be determined and tested by placing the tube in a saline filled beaker and testing the resistivity and capacitance of the tube before it is placed in the subject's brain or added to the model.

### Biological Assay

The second verification procedure is a biological assay to test stimulation of the motor cortex in patients who are having elective spinal surgeries that require tcMEPs as part of their surgical monitoring procedure. Effective current levels for stimulation in clinical patients may be established in this way. Since there is variation in the fine detail location of the motor cortex between individuals, it is advantageous to determine with precision the location of the target muscle as represented in the cortex.

Motor cortex localization is preferably determined by functional MRI (fMRI). The fMRI may be performed with the subject instructed to move his or her thumb (the abductor pollicic brevis muscle) to obtain precision location information of that muscle's representation in the motor cortex while the fMRI is being performed. The resulting imaged location can then be the target location for modeling of stimulation. The subject's MRI (and/or CT) is segmented as described. The subject's data are then received for GETs modeling for stimulation.

### Stimulation Site Algorithm

The best location for stimulating electrodes for targeting an identified motor cortex may be selected by the following algorithm. The target site may be identified. The computer may be programmed to systematically select and test for current density at the target site for each of the locations of the traditional 10-20 system for electrode placements on the head as current injection and extraction sites. In addition to the traditional 10-20 system sites, the eye lids, auditory canals and the nasal passage are preferably added, as they represent relevant avenues for bypassing the high resistivity of the skull. After the computer has grossly identified a pair of stimulation and extraction sites, the model may be refined in one centimeter increments around estimated sites. The new optimized sites are then preferably selected for use. The criteria the computer will use for target site evaluation is preferably the highest current achieved when a 10 Volt constant current square wave signal is modeled. The selected stimulation model is also examined for potential stray currents and preferably eliminated if they are judged to affect an area that might produce side effects (this is a safety procedure that is presently not possible).

### Surgical Stimulation

GETs modeling may be applied to multiple, e.g., 30, spinal surgery patients for verifying the efficacy of the GETs procedure by optimizing transcranial stimulation of the motor cortex through GETs modeling. The current needed to stimulate the same 30 patients is compared using the standard locations currently C3-C4 of the 10-20 system.

### TcMEP Recording Conditions

Anesthesia levels, blood pressure, and body temperature is preferably kept constant during the testing. No muscle relaxants are used for the preferred procedure, except during intubation. The low current levels allow stimuli to be presented through subdermal electrodes. During a patient's surgery, a patient may receive total intravenous anesthesia (TIVA) with propofol and narcotics to negate the inhibiting effect that traditional inhalation agents have on the motor cortex. These procedures are generally several hours long and testing can be done during a stable anesthetic regimen. The motor responses may be recorded from subdermal needle electrodes placed in the target muscle and recorded on a Cadwell Cascade intraoperative monitoring machine. Stimuli may be short duration square wave pulses presented through a constant current stimulator. The exact duration and intensity may be determined by the impedance properties predicted by the modeling.

The stimulus parameters may be identical between groups with a train of 6 square wave 100 μsec. pulses with a fix inter-stimulus duration and constant voltage. A minimum voltage and location may be determined by the model or the traditional sites found in the literature. The outcome variable may be the amplitude and duration of response as a reflection of the number of neurons activated in the fMRI identified loci of the motor cortex.

### Analysis

With CT/MRI imaging, analysis is preferably performed to determine if the improvement of the modeling is sufficient to justify the extra patient time and cost associated with the additional imaging involved in collecting a CT scan over and above a MRI. This can be accomplished with descriptive statistics and a T test. The second analysis will be to compare the electrode locations for stimulus site accuracy as reflected in the tcMEP responses observed in the operating room between traditional 10-20 locations cited in the literature and those predicted by the modeling. This analysis may be performed with a two way ANOVA.

Determining a precision β for a number of subjects involved in the between subjects testing is difficult, because there is no relevant history upon with to base our variance, but our experience in electrophysiology and surgery suggests that an N of 30 should be sufficient, because both of the conditions are to be tested on the same subjects.

### Risk Benefit Analysis and Alternate Methods

Electrical currents are advantageously reduced in a technique in accordance with a preferred embodiment as compared with conventional methods. In addition, already being performed surgeries can be used such that there is very little risk to subjects. The 2-D model effectively reduces involved currents, and the more realistic and computationally challenging 3-D model further reduces the currents used. These techniques advantageously improve the ability to stimulate the motor cortex in patients. This reduces the risk and improves the efficacy of the tcMEP procedure for surgical monitoring. A reduction of current densities to a level that allows for stimulation of awake patients is provided, and the same technique may be used to deliver brain stimulation in awake patient populations. A number of treatments that now involve invasive brain surgery are now available to patients at reduced cost and risk by utilizing the techniques of these preferred and alternative embodiments. These may include patients with refractory depression, epilepsy and chronic pain.

The modeling and resulting improved stimulation parameters in accordance with these embodiments may be used for tcMEP testing in the operating room environment. Transcranial electrical stimulation may be used in awake patients, as long as discomfort and pain involved are low enough, i.e., when current levels applied across the scalp are low enough as in accordance with a preferred embodiment. The advantageous reduction of stimulation levels permits reduction to levels of stimulation at less than 20 mA (constant voltage), and thus permits application of modeling to awake patients and those with refractory Parkinsonism disease. One of the advantages of GETs modeling is that, unlike physical models, the model may be continually improved as the quality of the imaging and computing capability improves. Advantageous results can also be achieved regarding other regions of the brain in addition to the motor cortex, and thus other medical conditions may be treated.

### Electrode within or through the Skull

The skin is a low resistance medium (approximately 230 ohms per cm) and the skull is very high resistance (approximately 1600 ohms per cm). When two or more electrodes are placed on the scalp and electrical energy is passed between them most of the energy t applied passes through the skin and relatively little goes into the brain. Thus the pain that is often felt when electrical current is applied to the head is really the result the electrical current that is passing through pain receptors in the scalp, and not to the stimulus that is reaching the brain. This can tend to limit amounts of electrical stimulus that can be applied to patients for therapy. This shunting of electrical energy though the scalp can be significantly reduced by placing electrodes within or through the skull and insulating the electrode from the scalp. In this manner electrical energy is directed away from the scalp and towards the brain.

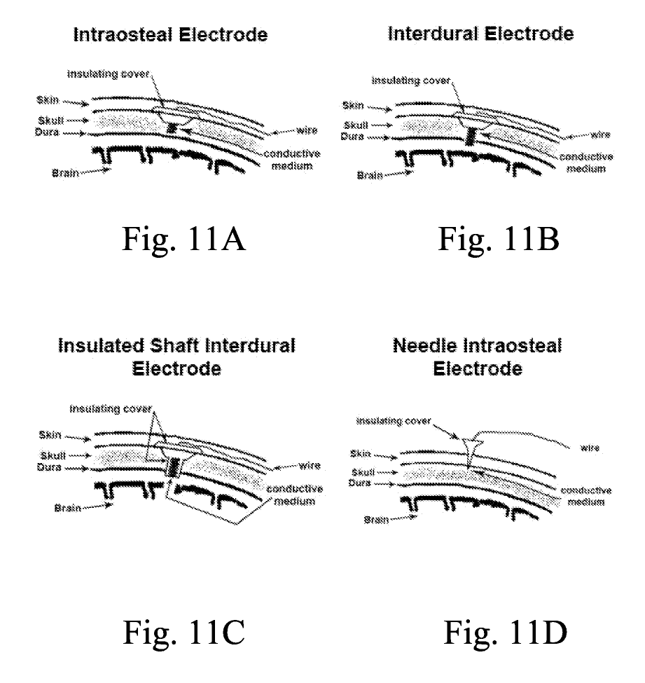

FIGS. 11a-11d illustrate electrode configurations in accordance with alternative embodiments, including intraosteal, interdural, insulated shaft interdural and needle intraosteal electrodes. Since the brain itself has no pain receptors, intra-osteal or trans-osteal electrodes properly insulated direct their stimulus toward the brain. Trans-osteal electrodes that touch the brain or dura may also have an insolating outer cover on the exposed portion that can prevent much of the electrical energy from being shunted through the cerebral spinal fluid and away from the brain surface that is directly under the electrode. Finally, the electrode may be flexible and or compressible so that it does not injure the underlying tissues when the brain moves in relation to the skull.

The present invention is not limited to the embodiments described above herein, which may be amended or modified without departing from the scope of the present invention, which is as set forth in the appended claims and structural and functional equivalents thereof.

In methods that may be performed according to preferred embodiments herein and that may have been described above and/or claimed below, the operations have been described in selected typographical sequences. However, the sequences have been selected and so ordered for typographical convenience and are not intended to imply any particular order for performing the operations.

In addition, all references cited above and below herein, in addition to the background and summary of the invention sections, are hereby incorporated by reference into the detailed description of the preferred embodiments as disclosing alternative embodiments and components. The following are incorporated by reference:

Amassian V E. Animal and human motor system neurophysiology related to intraoperative monitoring. In: Deletis V, Shels J, editors. Neurophysiology in Neurosurgery. Amsterdam: Academic Press, 2002:3-23;

Ary J P, Klein S A, Fender D H. Location of sources of evoked scalp potentials: corrections for skull and scalp thicknesses. IEEE Trans Biomed Eng 1981:28; 447-452;

Benar C G, Gotman J. Modeling of post-surgical brain and skull defects in the EEG inverse problem with the boundary element model. Clin Neurophysiol 2002; 113:48-56;

Ben-David, B., Haller G., Taylor P. Anterior spinal fusion complicated by paraplegia. A case report of false-negative somatosensory-evoked potential. Spine 1987, 12:536-9;

Berry M M, Standring S M, Bannister L H. Nervous system. In: Bannister L H, Berry M M, Collins P, Dyson M, Dussek J E, Ferguson M W J, editors. Gray's anatomy. The anatomical basis of medicine and surgery. New York: Churchill Livingstone, 1995:1191;

Bose B, Sestokas A K, Swartz D M. Neurophysiological monitoring of spinal cord function during instrumented anterior cervical fusion. Spine J 2004; 4:202-207;

Calachie B, Harris W, Brindle, F, Green B A, Landy H J. Threshold-level repetitive transcranial electrical stimulation for intraoperative monitoring of central motor conduction. J Neurosurg 2001; 95:161-168;

Chappa, K H Transcranial motor evoked potentials. Electromyogr. Clin. Neurophysiol. 1994;m34:15-21;

Cheney P D, Fetz E E, Palmer S S. Patterns of facilitation and suppression of antagonist forelimb muscles from motor cortex sites in the awake monkey. J Neurophysiol 985;53:805-820;

Comsol A B, FEMLAB User's Guide v. 3.0. Burlington, Mass.: Comsol, Inc, 2004;

Deletis V. Intraoperative neurophysiology and methodologies used to monitor the functional integrity of the motor system. In: Deletis V, Shels J, editors. Neurophysiology in Neurosurgery. Amsterdam: Academic Press, 2002: 25-51;

Eroglu, A., Solak, M., Ozem, I., and Aynaci, O. Stress hormones during the wake-up test in scoliosis surgery. J. Clin. Anesthesia 2003, 15: 15-18;

Ferree T C, Eriksen K J, Tucker D M. Regional head tissue conductivity estimation for improved EEG analysis. IEEE Trans Biomed Eng 2000; 47; 1584-1592;

Geddes L A, Baker L E. The specific resistance of biological material—a compendium of data for the biomedical engineer and physiologist. Med Biol Eng 1967; 271-293;

Ginsburge H. H., Shetter, A. G., Raudzens, P. A., Postoperative paraplegia with preserved intraopertive somatosensory evoked potentials. J. Neurosurg. 1985; 63:296-300;

Goncalves S I, de Munck J C, Verbunt J P A, Bijma F, Heethaar R M, da Silva F H. In vivo measurement of the brain and skull resistivities using an EIT-based method and realistic models for the head. IEEE Trans Biomed Eng 2003; 50:754-767;

Grimnes S. Martinsen O. G. Bioimpedance and Bioelectricity Basics Academic Press, San Diego, 2000;

Haghighi S S, Zhange R. Activation of the external anal and urethral sphincter muscles by repetitive transcranial cortical stimulation during spine surgery. J Clin Monit Comput 2004; 18:1-5;

Haueisen J, Ramon C. Influence of tissue resistivities on neuromagnetic fields and electric potentials studied with a finite element model of the head. IEEE Trans Biomed Eng 1997;44:727-735;

Henderson C J, Butler S R, Glass A. The localization of the equivalent dipoles of EEG sources by the application of electric field theory. Electroencephalogr Clin Neurophysiol 975;39:117-130;

Kavanagh R N, Darcey T M, Lehmann D, Fender D H. Evaluation of methods for the three-dimensional localization of electrical sources in the human brain. IEEE Trans Biomed Eng 1978; 25:421-429;

Knudsen, V Verification and use of a numerical computer program for simulation in bioimpedance. MSc thesis Dept. of Physics. Univ. Oslo, Norway;

Law S K. Thickness and resistivity variations over the upper surface of the human skull. Brain Topogr 1993;6:99-109;

Lesser, R. P., Raudzens, P., Luders, H., Nuwer, M. R., et al. Postoperative neurological deficits may occur despite unchanged intraoperative somatosensory evoked potentials. Ann. Neurol. 1986. 19:22-25;

Liu E H, Wong H K, Chia C P, Lim H J, Chen Z Y, Lee T L. Effects of isoflurane and propofol on cortical somatosensory evoked potentials during comparable depth of anaesthesia as guided by bispectral index. Br J Anaesth (in press);

MacDonald D B, Zayed Z A, Khoudeir I, Stigsby B. Monitoring scoliosis surgery with combined multiple pulse transcranial electric motor and cortical somatosensory-evoked potentials from the lower and upper extremities. Spine 2003; 28:194-203;

Mustain W. D. Kendig, R. I., Dissociation of neurogenic motor and somatosensory evoked potentials. A case report Spine. 1991; 16:851-3;

Nadeem M. Computation of electric and magnetic stimulation in human head using the 3-D impedance method. IEEE Trans Biomed Eng 2003; 50:900-907;

Nowinski W L, Bryan R N, Raghavan R. The electronic brain atlas. Multiplanar navigation of the human brain. New York: Thieme, 1997;

Oostendorp T F, Delbeke J, Stegeman D F. The conductivity of the human skull: results of in vivo and in vitro measurements. IEEE Trans Biomed Eng 2000; 47:1487-92;

Pelosi L, Lamb J, Grevitt M, Mehdian S M, Webb J K, Blumhardt L D. Combined monitoring of motor and somatosensory evoked potentials in orthopaedic spinal surgery. Clin Neurophysiol 2002; 113:1082-1091;

Rush S, Driscoll D A. Current distribution in the brain from surface electrodes. Anesth Analgesia 47; 717-723, 1968;

Schneider M. Effect of inhomogeneities on surface signals coming from a cerebral dipole source. IEEE Trans Biomed Eng 1974; 21:52-54;

Ubags L H, Kalkman C J, Been H D, Drummond J C. The use of a circumferential cathode improves amplitude of intraoperative electrical transcranial myogenic motor evoked responses. Anesth Analg 1996; 82:1011-1014;

Vauzelle, C., Stagnara, P., Jouvinroux, P. Functional monitoring of spinal cord activity during spinal surgery. Clin. Orthop. 1973; 93:173-8;

Zentner J. Non-invasive motor evoked potential monitoring during neurosurgical operations on the spinal cord. Neurosurg 1989; 24:709-712; and

US published patent applications nos. 2005/0244036, 2004/0215162, 2004/0102828 and 2002/0156372; and

U.S. Pat. Nos. 6,608,628, 6,763,140, 5,750,895, 5,805,267, 6,106,466, 6,236,738, 6,476,804, 6,959,215, 6,330,446, 7,010,351, 6,463,317, 6,322,549, 6,248,080, 6,230,049, 6,006,124, 6,045,532, 6,916,294, 6,937,905, 6,675,048, 6,607,500, 6,560,487, 6,324,433, 6,175,769, 5,964,794, 5,725,377, 5,255,692, 4,611,597, 4,421,115, and 4,306,564.

What is claimed is:

1. A method of determining an optimal transcranial or intracranial, or other trans-tissue application of electrical energy for therapeutic treatment, comprising:
(a) obtaining MRI or CAT scan data, or both, of a subject brain or other body tissue;
(b) assigning different anisotropic electrical values to portions of the subject brain or other body tissue based on the data;
(c) selecting electrode sites; and
(d) calculating, based on the assigning and selecting, one or more applied electrical inputs for optimal therapeutic application of transcranial or intracranial or other trans-tissue electricity.

2. The method of claim 1, wherein the assigning comprises:
(i) segmenting the subject brain by defining tissue compartment boundaries between, and one or more electricalcharacteristics to, said portions of the subject brain;
(ii) implementing a finite element model by defining a mesh of grid elements for the subject brain; and
(iii) ascribing vector resistance values to each of the grid elements based on the segmenting.

3. The method of claim 2, wherein the electrical inputs comprise applied voltages, currents, energies, pulse shapes, pulse durations, pulse heights, or number of pulses per pulse train, or combinations thereof, and the electricity comprises current.

4. The method of claim 3, further comprising resolving peaks within respective gray scale data corresponding to two or more brain or other body tissues.

5. The method of claim 2, wherein the segmenting comprises discriminating two or more of the following organic brain substances: cerebral spinal fluid, white matter, blood, skin, gray matter, soft tissue, cancellous bone and compact bone.

6. The method of claim 5, wherein the discriminating comprises resolving peaks within respective gray scale data corresponding to the two or more organic brain substances.

7. The method of claim 2, wherein the ascribing further comprises inferring anisotropies for the electrical values of the grid elements.

8. The method of claim 1, wherein the electrical values comprise resistivities, conductivities, capacitances, impedances, applied energies or charges, or combinations thereof.

9. The method of claim 1, wherein the electrical values comprise resistivities.

10. The method of claim 1, wherein the data comprises a combination of two or more types of MRI or CAT scan data, or both.

11. The method of claim 1, wherein the data comprises a combination of two of more of T1, T2 and PD MRI data.

12. The method of claim 1, wherein the data comprises three-dimensional data.

13. The method of claim 1, wherein the selecting comprises disposing the electrodes within the skull tissue.

14. The method of claim 1, wherein the selecting comprises disposing the electrodes through the skull proximate to or in contact with the dura.

15. The method of claim 1, wherein the selecting comprises disposing the electrodes in a shallow transdural location.

16. The method of claim 1, wherein the selecting comprises utilizing a screw mounted electrode within or through the skull tissue.

17. A method of determining an optimal transcranial or intracranial application of electrical energy for therapeutic treatment, comprising:
(a) obtaining a combination of two or more types of three-dimensional MRI or CAT scan data, or both, of a subject brain;
(b) assigning different electrical values to portions of the subject brain based on the data;
(c) selecting electrode sites including disposing at least one electrode at least partially through the skull; and
(d) calculating, based on the assigning and selecting, one or more applied electrical inputs for optimal therapeutic application of transcranial or intracranial electricity.

18. The method of claim 17, wherein the assigning comprises:
(i) segmenting the subject brain by defining tissue compartment boundaries between, and one or more anisotropic electrical resistance characteristics to, said portions of the subject brain;
(ii) implementing a finite element model by defining a mesh of grid elements for the subject brain; and
(iii) ascribing vector electrical values to each of the grid elements based on the segmenting.

19. The method of claim 17, wherein the electrical inputs comprise applied voltages, currents, energies, pulse shapes, pulse durations, pulse heights, or number of pulses per pulse train, or combinations thereof, and the electricity comprises current.

20. The method of claim 17, wherein the segmenting comprises discriminating two or more of the following organic brain substances: cerebral spinal fluid, white matter, blood, skin, gray matter, soft tissue, cancellous bone and compact bone.

21. The method of claim 17, wherein the data comprises a combination of two of more of T1, T2, DT and PD MRI data.

22. The method of claim 17, wherein the selecting comprises disposing at least one electrode through the skull proximate to or in contact with the dura.

23. The method of claim 17, wherein the selecting comprises disposing at least one electrode in a shallow transdural location.

24. The method of claim 17, wherein the selecting comprises utilizing a screw mounted electrode within or through the skull tissue.

25. A method of determining an optimal transcranial or intracranial or other trans-tissue application of electrical energy for therapeutic treatment, comprising:
(a) obtaining MRI or CAT scan data, or both, of a subject brain or other body tissue;
(b) segmenting the subject brain by defining tissue compartment boundaries between, and one or more electrical characteristics to, said portions of the subject brain or other body tissue;
(c) implementing a finite element model by defining a mesh of grid elements for the subject brain or other body tissue;
(d) ascribing electrical values to each of the grid elements based on the segmenting;
(e) selecting electrode sites; and
(f) calculating, based on the ascribing and selecting, one or more applied electrical inputs for optimal therapeutic application of transcranial or intracranial or other trans-tissue current.

26. The method of claim 25, wherein the electrical values comprise vector resistance values and the electrical characteristics comprises anisotropies.

27. The method of claim 25, wherein the electrical inputs comprise applied voltages, currents, energies, pulse shapes, pulse durations, pulse heights, or number of pulses per pulse train, or combinations thereof.

28. The method of claim 25, wherein the segmenting comprises discriminating two or more of the following organic brain substances: cerebral spinal fluid, white matter, blood, skin, gray matter, soft tissue, cancellous bone, eye fluid, cancerous tissue, inflammatory tissue, ischemic tissue and compact bone.

29. The method of claim 25, wherein the ascribing further comprises inferring anisotropies for the resistance values of the grid elements.

30. The method of claim 25, wherein the data comprises a combination of two or more types of MRI or CAT scan data, or both.

31. The method of claim 25, wherein the data comprises a combination of two of more of T1, T2, DT and PD MRI data.

32. The method of claim 25, wherein the data comprises three-dimensional data.

33. A method of determining an optimal transcranial or intracranial or other trans-tissue application of electrical energy for therapeutic treatment based on MRI or CAT scan data, or both, of a subject brain or other body tissue, and different anisotropic electrical values assigned to portions of the subject brain or other body tissue based on the data, the method comprising:
(a) selecting electrode sites; and
(b) calculating, based on the assigned anisotropic electrical values and the selecting, one or more applied electrical inputs for optimal therapeutic application of transcranial or intracranial or other trans-tissue current.

34. The method of claim 33, wherein the anisotropic values are assigned based on:
(i) segmenting the subject brain by defining tissue compartment boundaries between, and one or more electrical characteristics to, said portions of the subject brain;
(ii) implementing a finite element model by defining a mesh of grid elements for the subject brain; and
(iii) ascribing vector resistance values to each of the grid elements based on the segmenting.

35. The method of claim 34, wherein the segmenting comprises discriminating two or more of cerebral spinal fluid, white matter, blood, skin, gray matter, soft tissue, cancellous bone, eye fluid, cancerous tissue, inflammatory tissue, ischemic tissue, and compact bone.

36. The method of claim 35, wherein the discriminating comprises resolving peaks within respective gray scale data corresponding to two or more brain or other body tissues.

37. A method of determining an optimal transcranial or intracranial or other trans-tissue application of electrical energy for therapeutic treatment based on obtaining MRI or CAT scan data, or both, of a subject brain or other body tissue, and electrical values ascribed to grid elements of a mesh defined by implementing a finite element model for a subject brain or other body tissue, and by segmenting the subject brain or other body tissue by defining tissue compartment boundaries between, and one or more electrical characteristics to, said portions of the subject brain or other body tissue, and by implementing a finite element model by defining a mesh of grid elements for the subject brain, and ascribing electrical values to each of the grid elements based on the segmenting, the method comprising:
(a) selecting electrode sites; and
(b) calculating, based on the ascribed electrical values and selecting, one or more applied electrical values for optimal therapeutic application of transcranial or intracranial or other trans-tissue current.

38. The method of claim 37, wherein the electrical values comprise vector resistance values and the electrical characteristics comprises anisotropies.

39. The method of claim 37, wherein the segmenting comprises discriminating eye fluid and cerebral spinal fluid, or two or more of cerebral spinal fluid, white matter, blood, skin, gray matter, soft tissue, cancellous bone, eye fluid, cancerous tissue, inflammatory tissue, ischemic tissue, and compact bone.

40. The method of claim 37, wherein the ascribing further comprises inferring anisotropies for the resistance values of the grid elements.

41. One or more processor readable storage devices having processor readable code embodied thereon, said processor readable code for programming one or more processors to perform a method of determining an optimal transcranial or intracranial or other trans-tissue application of electrical energy for therapeutic treatment, the method comprising:
(a) obtaining MRI or CAT scan data, or both, of a subject brain or other body tissue;
(b) assigning different anisotropic electrical values to portions of the subject brain or other body tissue based on the data;
(c) selecting electrode sites; and
(d) calculating, based on the assigning and selecting, one or more applied electrical inputs for optimal therapeutic application of transcranial or intracranial or other trans-tissue electricity.

42. The one or more storage devices of claim 41, wherein the assigning comprises:
(i) segmenting the subject brain by defining tissue compartment boundaries between, and one or more electrical characteristics to, said portions of the subject brain;
(ii) implementing a finite element model by defining a mesh of grid elements for the subject brain; and
(iii) ascribing vector electrical values to each of the grid elements based on the segmenting.

43. The one or more storage devices of claim 42, wherein the electrical inputs comprise applied voltages, currents, energies, pulse shapes, pulse durations, pulse heights, or number of pulses per pulse train, or combinations thereof, and the electricity comprises current.

44. The one or more storage devices of claim 43, wherein the discriminating comprises resolving peaks within respective gray scale data corresponding to two or more brain or other body tissues.

45. The one or more storage devices of claim 43, wherein the segmenting comprises discriminating two or more of the following: cerebral spinal fluid, white matter, blood, skin, gray matter, soft tissue, cancellous bone, eye fluid, cancerous tissue, inflammatory tissue, ischemic tissue and compact bone.

46. The one or more storage devices of claim 45, wherein the discriminating comprises resolving peaks within respective gray scale data corresponding to the two or more brain or other body tissues.

47. The one or more storage devices of claim 42, wherein the ascribing further comprises inferring anisotropies for the resistance values of the grid elements.

48. The one or more storage devices of claim 41, wherein the electrical values comprise conductivities, resistivities, capacitances, impedances, applied energies, power, charge, or combinations thereof.

49. The one or more storage devices of claim 41, wherein the electrical values comprise resistivities.

50. The one or more storage devices of claim 41, wherein the data comprises a combination of two or more types of MRI or CAT scan data, or both.

51. The one or more storage devices of claim 41, wherein the data comprises a combination of two of more of T1, T2, DT and PD MRI data.

52. The one or more storage devices of claim 41, wherein the data comprises three-dimensional data.

53. The one or more storage devices of claim 41, wherein the selecting comprises disposing the electrodes within the skull tissue.

54. The one or more storage devices of claim 41, wherein the selecting comprises disposing the electrodes through the skull proximate to or in contact with the dura.

55. The one or more storage devices of claim 41, wherein the selecting comprises disposing the electrodes in a shallow transdural location.

56. The one or more storage devices of claim 41, wherein the selecting comprises utilizing a screw mounted electrode within or through the skull tissue.

57. One or more processor readable storage devices having processor readable code embodied thereon, said processor readable code for programming one or more processors to perform a method of determining an optimal transcranial or intracranial application of electrical energy for therapeutic treatment, the method comprising:
(a) obtaining a combination of two or more types of three-dimensional MRI or CAT scan data, or both, of a subject brain;
(b) assigning different electrical values to portions of the subject brain based on the data;
(c) selecting electrode sites including disposing at least one electrode at least partially through the skull; and
(d) calculating, based on the assigning and selecting, one or more applied electrical inputs for optimal therapeutic application of transcranial or intracranial current.

58. The one or more storage devices of claim 57, wherein the assigning comprises:
(i) segmenting the subject brain by defining tissue compartment boundaries between, and one or more anisotropic electrical characteristics to, said portions of the subject brain;
(ii) implementing a finite element model by defining a mesh of grid elements for the subject brain; and
(iii) ascribing vector electrical values to each of the grid elements based on the segmenting.

59. The one or more storage devices of claim 57, wherein the electrical inputs comprise applied voltages, currents, energies, pulse shapes, pulse durations, pulse heights, or number of pulses per pulse train, or combinations thereof.

60. The one or more storage devices of claim 57, wherein the segmenting comprises discriminating two or more of the following: cerebral spinal fluid, white matter, blood, skin, gray matter, soft tissue, cancellous bone, eye fluid, cancerous tissue, inflammatory tissue, ischemic tissue, and compact bone.

61. The one or more storage devices of claim 57, wherein the data comprises a combination of two of more of T1, T2, DT and PD MRI data.

62. The one or more storage devices of claim 57, wherein the selecting comprises disposing at least one electrode through the skull proximate to or in contact with the dura.

63. The one or more storage devices of claim 57, wherein the selecting comprises disposing at least one electrode in a shallow transdural location.

64. The one or more storage devices of claim 57, wherein the selecting comprises utilizing a screw mounted electrode within or through the skull tissue.

65. One or more processor readable storage devices having processor readable code embodied thereon, said processor readable code for programming one or more processors to perform a method of determining an optimal transcranial or intracranial or other trans-tissue application of electrical energy for therapeutic treatment, the method comprising:
(a) obtaining MRI or CAT scan data, or both, of a subject brain or other body tissue;
(b) segmenting the subject brain or other body tissue by defining tissue compartment boundaries between, and one or more electrical characteristics to, said portions of the subject brain or other body tissue;
(c) implementing a finite element model by defining a mesh of grid elements for the subject brain or other body tissue;
(d) ascribing electrical values to each of the grid elements based on the segmenting;
(e) selecting electrode sites; and
(f) calculating, based on the assigning and selecting, one or more applied electrical inputs for optimal therapeutic application of transcranial or intracranial or other trans-tissue current.

66. The one or more storage devices of claim 65, wherein the electrical values comprise vector resistance values and the electrical characteristics comprises anisotropies.

67. The one or more storage devices of claim 65, wherein the electrical inputs comprise applied voltages, currents, energies, pulse shapes, pulse durations, pulse heights, or number of pulses per pulse train, or combinations thereof.

68. The one or more storage devices of claim 65, wherein the segmenting comprises discriminating two or more of the following: cerebral spinal fluid, white matter, blood, skin, gray matter, soft tissue, cancellous bone, eye fluid, cancerous tissue, inflammatory tissue, ischemic tissue, and compact bone.

69. The one or more storage devices of claim 65, wherein the ascribing further comprises inferring anisotropies for the resistance values of the grid elements.

70. The one or more storage devices of claim 65, wherein the data comprises a combination of two or more types of MRI or CAT scan data, or both.

71. The one or more storage devices of claim 65, wherein the data comprises a combination of two of more of T1, T2, DT and PD MRI data.

72. The one or more storage devices of claim 65, wherein the data comprises three-dimensional data.

73. One or more processor readable storage devices having processor readable code embodied thereon, said processor readable code for programming one or more processors to perform a method of determining an optimal transcranial or intracranial or other trans-tissue application of electrical energy for therapeutic treatment based on MRI or CAT scan data, or both, of a subject brain or other body tissue, and different anisotropic electrical values assigned to portions of the subject brain or other body tissue based on the data, the method comprising:
(a) selecting electrode sites; and
(b) calculating, based on the assigned anisotropic values and the selecting, one or more applied electrical inputs for optimal therapeutic application of transcranial or intracranial or other trans-tissue current.

74. The one or more storage devices of claim 73, wherein the anisotropic values are assigned based on:
(i) segmenting the subject brain by defining tissue compartment boundaries between, and one or more electrical characteristics to, said portions of the subject brain;
(ii) implementing a finite element model by defining a mesh of grid elements for the subject brain; and
(iii) ascribing vector electrical values to each of the grid elements based on the segmenting.

75. The one or more storage devices of claim 74, wherein the segmenting comprises discriminating two or more of cerebral spinal fluid, white matter, blood, skin, gray matter, soft tissue, cancellous bone, eye fluid, cancerous tissue, inflammatory tissue, ischemic tissue, and compact bone.

76. The one or more storage devices of claim 75, wherein the discriminating comprises resolving peaks within respective gray scale data corresponding to the two or more brain or other body tissues.

77. One or more processor readable storage devices having processor readable code embodied thereon, said processor readable code for programming one or more processors to perform a method of determining an optimal transcranial or intracranial or other trans-tissue application of electrical energy for therapeutic treatment based on obtaining MRI or CAT scan data, or both, of a subject brain or other body tissue, and electrical values ascribed to grid elements of a mesh defined by implementing a finite element model for a subject brain or other body tissue, and by segmenting the subject brain or other body tissue by defining tissue compartment boundaries between, and one or more electrical characteristics to, said portions of the subject brain or other body tissue, and by implementing a finite element model by defining a mesh of grid elements for the subject brain or other body tissue, and ascribing electrical values to each of the grid elements based on the segmenting, the method comprising:
(a) selecting electrode sites; and
(b) calculating, based on the ascribed electrical values and selecting, one or more applied electrical inputs for optimal therapeutic application of transcranial or intracranial or other trans-tissue current.

78. The one or more storage devices of claim 77, wherein the electrical values comprise vector resistance values and the electrical characteristics comprises anisotropies.

79. The one or more storage devices of claim 77, wherein the segmenting comprises discriminating two or more of cerebral spinal fluid, white matter, blood, skin, gray matter, soft tissue, cancellous bone, eye fluid, cancerous tissue, inflammatory tissue, ischemic tissue, and compact bone.

80. The one or more storage devices of claim 77, wherein the ascribing further comprises inferring anisotropies for the electrical values of the grid elements.
